(19) United States
(12) Patent Application Publication
(10) Pub. No.: US 2006/0241653 A1
(43) Pub. Date: Oct. 26, 2006

(54) Surgical hook instrument for gastric band closing

(76) Inventors: Daniel B. Jones, Wayland, MA (US); Jerry R. Griffiths, Pembroke, MA (US); Francis J. Di Francesco, Foxboro, MA (US)

Correspondence Address: AKC PATENTS, 215 GROVE ST., NEWTON, MA, 02466, US

(21) Appl. No.: 11/315,822

(22) Filed: Dec. 22, 2005

Related U.S. Application Data
(60) Provisional application No. 60/650,284, filed on Feb. 4, 2005.
(60) Provisional application No. 60/650,290, filed on Feb. 4, 2005.
(60) Provisional application No. 60/670,111, filed on Apr. 11, 2005.

(51) Int. Cl. A61B 17/42

(52) U.S. Cl. 606/125

(57) ABSTRACT

An endoscopic surgical hook instrument is used in minimally invasive laparoscopic surgery for closing a gastric band having a buckle end a free end. The hook instrument includes a flat hook end that is placed underneath or on top of a pull tab that extends from the buckle end and through a hole of the pull tab. The hook instrument holds the buckle end securely while a pusher instrument is used to push the free end of the gastric band through the buckle.

### CROSS REFERENCE TO RELATED CO-PENDING APPLICATIONS

This application claims the benefit of U.S. provisional application Ser. No. 60/650,284 filed on Feb. 4, 2005 and entitled SURGICAL HOOK INSTRUMENT FOR GASTRIC BAND CLOSING which is commonly assigned and the contents of which are expressly incorporated herein by reference.

This application is also related to U.S. provisional application Ser. No. 60/650,290 filed on Feb. 4, 2005 and entitled SURGICAL ROTARY CAPTURE INSTRUMENT FOR GASTRIC BAND CLOSING which is commonly assigned and the contents of which are expressly incorporated herein by reference.

This application is also related to U.S. provisional application Ser. No. 60/670,111 filed on Apr. 11, 2005 and entitled GASTRIC BAND INSERTION INSTRUMENT which is commonly assigned and the contents of which are expressly incorporated herein by reference.

### FIELD OF THE INVENTION

The present invention relates to an endoscopic surgical hook instrument, and more particularly to a surgical hook instrument used in minimally invasive laparoscopic surgery for closing a gastric band.

### BACKGROUND OF THE INVENTION

One method of controlling the intake of food in an obese person is to place an adjustable restriction band 10 around the upper stomach 20, shown in FIG. 1. This creates a new small stomach pouch in the upper stomach 20 for holding a small amount of food and leaves the larger part of the stomach below the band so the stomach volume available for holding food is reduced. The band also controls the stoma, i.e., stomach outlet, between the upper stomach and the lower stomach 30. The size of the stoma regulates the flow of food from the upper stomach to the lower stomach. When the stoma is small the patient feels full sooner and has a feeling of satiety that lasts longer.

One specific type of an adjustable restriction band 10 is the LAP-BAND system manufactured by INAMED Corporation, shown in FIG. 2A and FIG. 2B. The LAP-BAND system is described in U.S. Pat. No. 5,601,604, the contents of which are incorporated herein by reference. Referring to FIG. 2A, the gastric band 10 of the LAP-BAND system includes a body portion 11 a head portion 12 and a tail portion 13. The head portion 12 has a buckle 19 with a pull tab 18 and the pull tab 18 has a hole 18a for receiving a post. The tail portion 13 has a tube 14 extending from one end, a triangular shaped member 13a and a conical shaped barb 13b. Tube 14 is in communication with an inflatable member 16 of the inner surface 15 of the body portion 11. The inflatable member 16 is gradually inflated by injecting a saline solution through the tube 14. The inflated member 16 presses against and constricts the stomach wall underlying the band 10. This results in decreasing the diameter of the stoma. The amount of the injected solution controls the size of the inflated member 16 and accordingly the diameter of the stoma.

During a minimally invasive laparoscopic surgical procedure, the tube 14 of the gastric band 10 is pushed through a laparoscopic cannula and is inserted in the patient's abdomen. The gastric band 10 is then placed around the patient's upper stomach and the tail portion 13 is inserted into the buckle 19 thereby forming a ring structure around the upper stomach. The triangular shaped member 13a of the tail portion 13 interlocks with the buckle 19 and prevents the tube 14 from slipping backwards. The process of inserting the tail portion 13 into the buckle 19 requires simultaneously grasping the buckle 19 and threading and pushing the tube 14 through the buckle conduit 19a. Minimally invasive tools are used for performing these two mechanical manipulations needed for tightening the gastric band around the upper stomach.

A prior art combination tool for grasping the pull tab 18 and pushing the tube 14 is described in U.S. Pat. No. 5,658,298. This prior art tool includes two elongated slidably mounted cylindrical members and a handpiece. At the distal end of the first cylindrical member there is a post extending downwards that is dimensioned to engage the hole 18a of the tab 18 from the top side, shown in FIG. 6 of U.S. Pat. No. 5,658,298. The second cylindrical member, which is slidably mounted with respect to the first cylindrical member, has mounted on its distal end a seat or a fork, shown in FIG. 3A of U.S. Pat. No. 5,658,298. This fork is dimensioned to capture protuberances such as the conical barb 13b at the tube end of the band 10 and push the tube 14 through the buckle conduit 19a, while the first cylindrical member engages and pulls on the pull tab 18. Surgeons performing this type of laparoscopic surgery have encountered the problems of the post unintentionally slipping out of the hole 18a and the fork unintentionally releasing the tube end of the band 10 during the band tightening procedure. This requires regrasping the tube end of the band 10 and the tab 18 several times during the procedure, which increases both the operation time and the complexity of the operation.

Accordingly there is a need for an improved grasping and pushing tool used in tightening a band having a buckle end and a free end that does not disengage unintentionally and provides better stability and control during the tightening procedure.

### SUMMARY OF THE INVENTION

In general, in one aspect, the invention features an endoscopic surgical hook tool used in minimally invasive surgery for grasping and tightening a ligature band. The ligature band comprises an elongated strap having a buckle end and a distal end. The buckle end has an aperture and a pull tab having a hole. The elongated strap is configured to encircle an internal organ and the distal end is configured to pass through and lockingly engage the aperture thereby tightening the ligature band around the internal organ. The hook tool includes an elongated shaft having a hook end and a handle. The hook end is configured to engage the hole and pull the pull tab in a first direction while the distal end is threaded and pushed through the aperture, opposite to the first direction. The hook end comprises a flat portion and a bend portion extending from the flat portion. The flat portion is configured to slide along a flat surface of the pull tab and the bend portion has an inner surface radius matching a radius of the hole.

Implementations of this aspect of the invention may include one or more of the following features. The elongated shaft is dimensioned to enter one end, pass through and extend beyond the other end of the cannula. The handle is configured to provide tactile control of the hook end orientation. The handle comprises a cylindrical body having first and second side indentations opposite to each other and a thumb indentation on a top surface of the cylindrical body, the thumb indentation being aligned with the bend portion of the hook end. The hook end further comprises an extension portion extending from the bend portion, running parallel to the flat portion and pointing towards the handle. The internal organ may be a stomach, artery, intestines, heart, lung, pancreas, kidney, bone or liver. In general, in another aspect, the invention features an endoscopic surgical hook tool used in minimally invasive surgery through a cannula for grasping and pulling an elongated strap having a pull tab. The hook tool includes an elongated shaft having a hook end and a handle. The hook end comprises a flat portion and a bend portion extending from the flat portion and the flat portion is configured to slide along a flat surface of the pull tab and the bend portion is configured to engage and grasp a hole on the pull tab. The bend portion has an inner surface radius matching a radius of the hole.

In general, in another aspect, the invention features an endoscopic surgical instrument used in minimally invasive surgery through a cannula for grasping and tightening a ligature band around an internal organ. The ligature band comprises an elongated strap having a buckle end and a distal end. The buckle end has an aperture and a pull tab having a hole thereon. The elongated strap is configured to encircle the internal organ and the distal end is configured to pass through and lockingly engage the aperture thereby tightening the ligature band around the internal organ. The surgical instrument includes a hook tool configured to engage the hole and pull the pull tab in a first direction while the distal end is threaded and pushed through the aperture, opposite to the first direction. The surgical instrument also includes a pusher tool configured to engage a protuberance of the distal end and push the distal end through the aperture after it has been threaded through it. The hook tool comprises a hook having a flat portion and a bend portion extending from the flat portion and the flat portion is configured to slide along a flat surface of the pull tab and the bend portion has an inner surface radius matching a radius of the hole. The hook tool and the pusher tool apply opposing forces on the ligature band for tightening the ligature band around the internal organ. The hook tool may be used in other non endoscopic procedures.

In general, in another aspect, the invention features a method for tightening a ligature band around an internal organ via minimally invasive surgery. The method includes providing a ligature band comprising an elongated strap having a buckle end and a distal end. The buckle end has an aperture and a pull tab having a hole thereon. Next, inserting the ligature band into a patient's body through a minimally invasive cannula and encircling the internal organ with the elongated strap. Next, inserting a surgical hook tool through the cannula. The hook tool comprises an elongated shaft having a hook end and a handle and the hook end comprises a flat portion and a bend portion extending from the flat portion and the flat portion is configured to slide along a flat surface of the pull tab and the bend portion has an inner surface radius matching a radius of the hole. Next, engaging the hole with the hook end and then pulling the pull tab in a first direction with the hook tool while pushing the distal end through the aperture, opposite to the first direction.

Among the advantages of this invention may be one or more of the following. The bend portion of the hook end together with the extension and the flat portion form a C-shaped hook that grasps securely the pull tab. The risk of unintentional disengagement is very low. The extension helps prevent slippage of the hook out of the hole. The inner surface of the bend portion is formed with a radius that matches the inside of the hole in the pull tab. This distributes the pulling force uniformly around the hole and prevents damaging of the pull tab during pulling. The flat portion of the hook end allows the hook end to slide easily underneath or above the pull tab.

The details of one or more embodiments of the invention are set forth in the accompanying drawings and description below. Other features, objects and advantages of the invention will be apparent from the following description of the preferred embodiments, the drawings and from the claims.

### BRIEF DESCRIPTION OF THE DRAWINGS

Referring to the figures, wherein like numerals represent like parts throughout the several views:

FIG. 1 is a side view of a stomach with an adjustable gastric band around the upper part of the stomach;

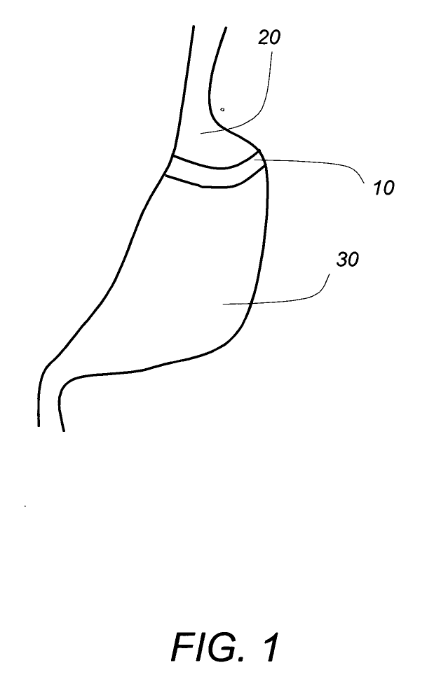

FIG. 2A is a perspective view of an open LAP-BAND gastric band;

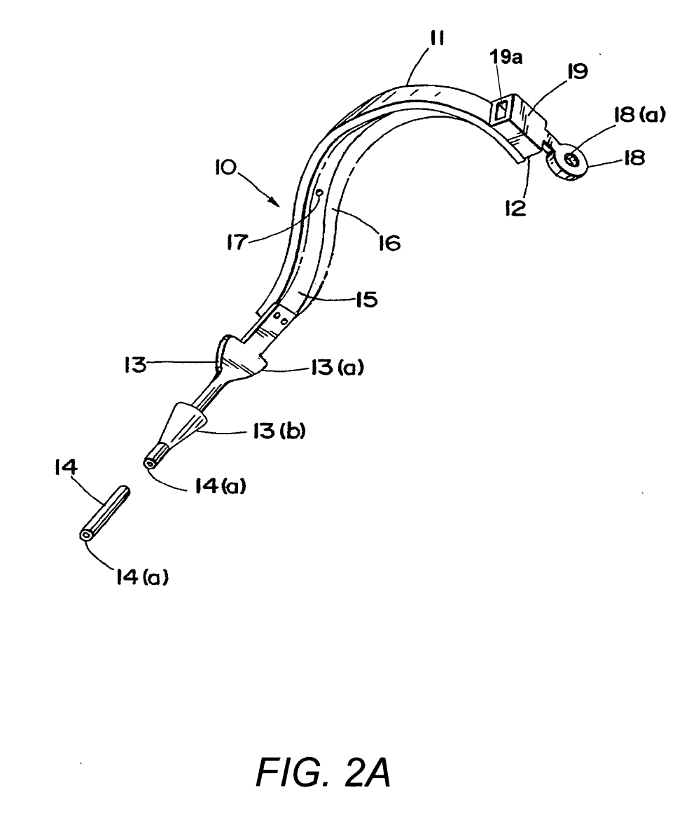

FIG. 2B is the gastric band of FIG. 2A in a closed position forming a ring structure;

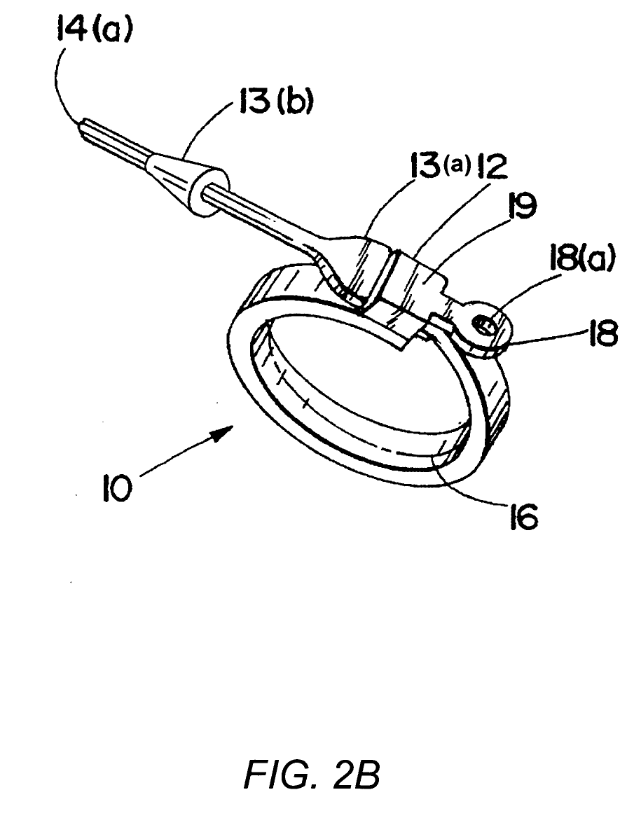

FIG. 3 is a perspective view of the endoscopic hook instrument of this invention;

FIG. 4 is a perspective view of the hook end of the instrument of FIG. 3;

FIG. 5 is a side view of the hook of FIG. 4;

FIG. 6 is a top view of the hook of FIG. 4;

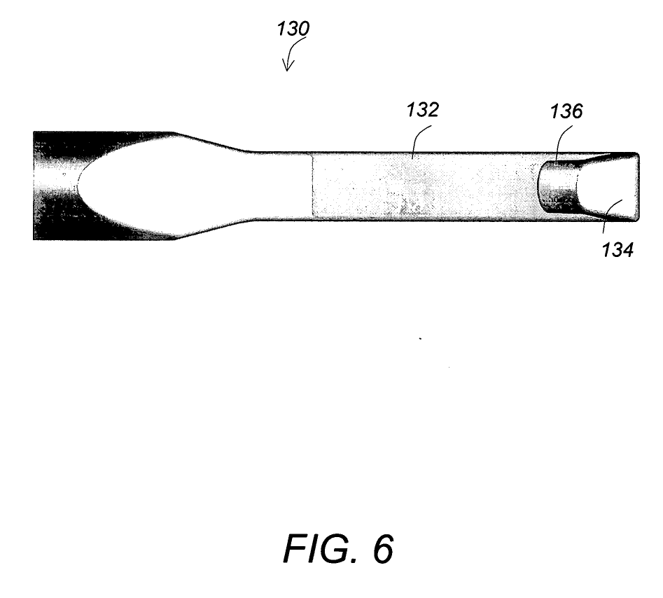

FIG. 7 is perspective view of the handle of the instrument of FIG. 3;

FIG. 8 is side view of the hook end of the instrument of FIG. 3 engaging the hole in the tab of the buckle of the gastric band of FIG. 2A; and

FIG. 9 is a perspective view of the hook end of the instrument of FIG. 3 engaging the hole in the tab of the buckle of the gastric band of FIG. 2A.

### DETAILED DESCRIPTION OF THE INVENTION

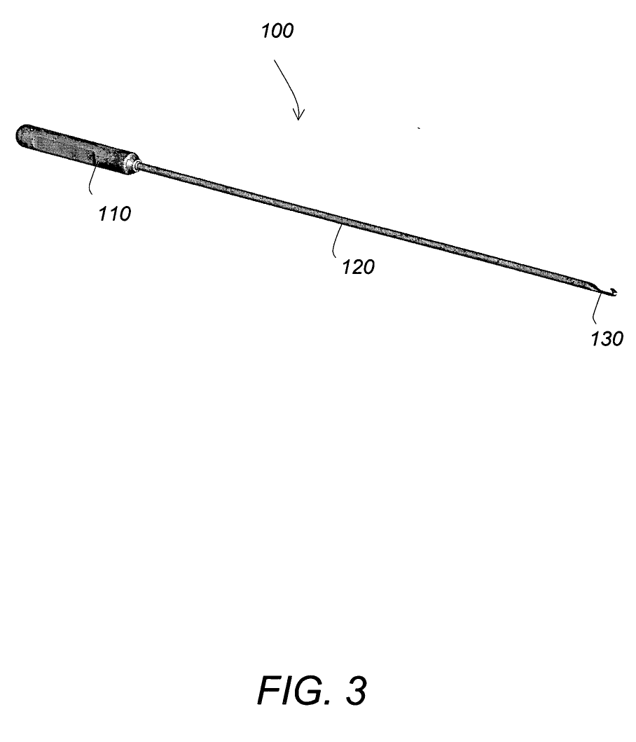

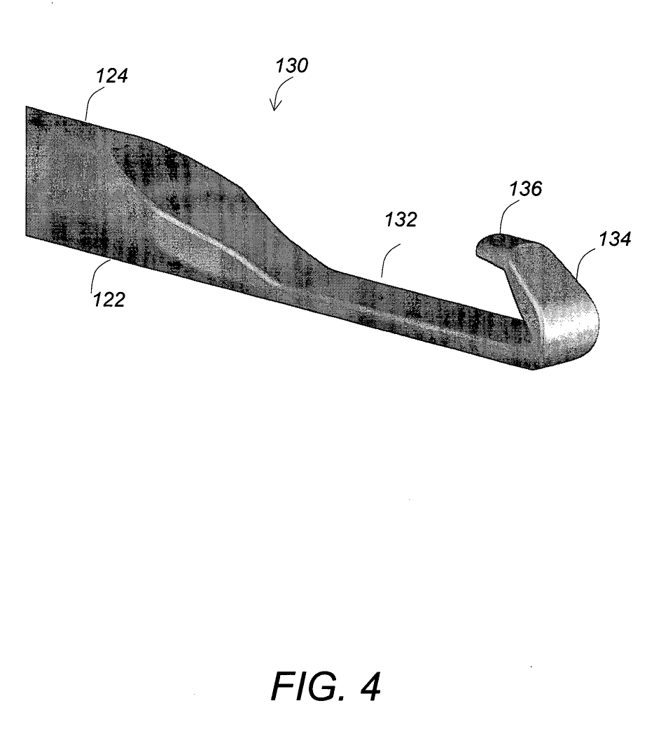

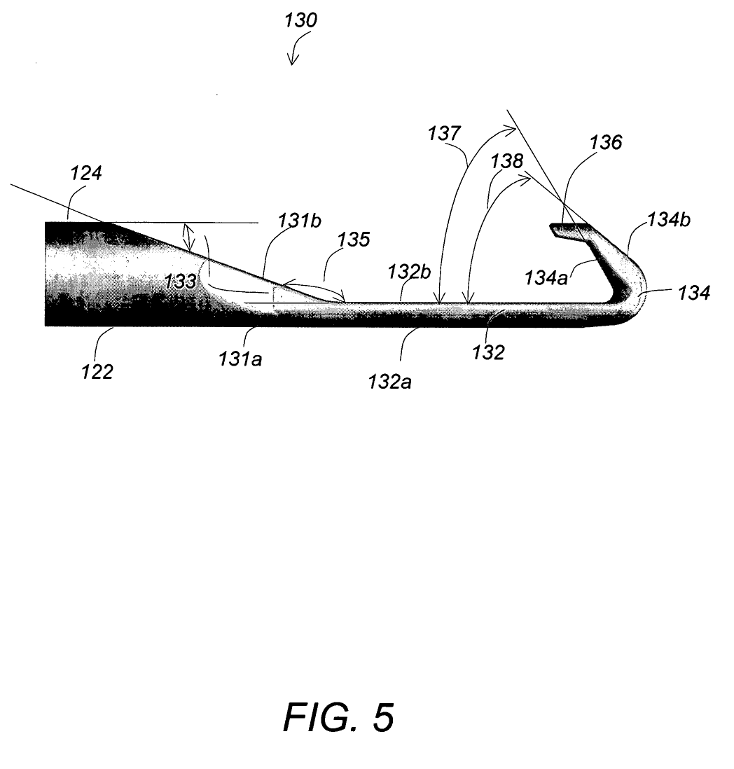

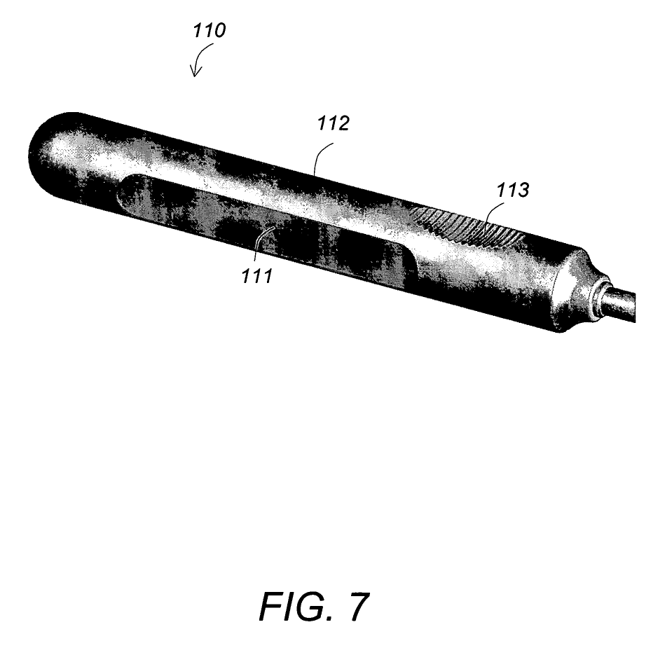

Referring to FIG. 3, a surgical hook instrument 100 includes a handle 110, an elongated shaft 120 and a hook end 130. The handle 110 features a tactile control of the hook end. Referring to FIG. 7 the handle 110 features a cylindrical body having a diameter of 15.9 mm and a length of 11.4 cm and it includes two side indentations 111, 112 opposite to each other and a thumb indentation 113 on the top surface. The elongated shaft 120 has a cylindrical shape and is dimensioned to fit through a laparoscopic cannula for minimally invasive surgery. In one example, the elongated shaft 120 has a length of 45 cm and a diameter of 5 mm. Referring to FIG. 4 and FIG. 5, the hook end 130 includes an angled portion, a flat portion 130 a bend portion 134 and an extension 136. The angled portion 131 has a flat bottom surface 131a and a downwards slopped top surface 131b forming an angle 133 with the top surface 124 of the elongated shaft 120. The flat portion 132 has a flat top surface 132b and a flat bottom surface 132a that extends continuously from the bottom surface 122 of the elongated shaft 120 and the bottom surface 131a of the angled portion 131. The top surface 132b of the flat portion 132 forms an angle 135 with the top surface 131b of the angled portion 131. Angles 133 and 135 are supplementary to each other, i.e., their sum is 180 degrees. In one example, angle 133 is 20 degrees and angle 135 is 160 degrees. In one example, the flat portion 132 has a thickness of 1.3 mm, a width of 3.3 mm and a length of 13.4 mm. The bend portion 134 extends from the flat portion 132 and is bend upwards and towards the handle 110. The inner surface 134a of the bend portion 134 forms an angle 137 with the top surface 132b of the flat portion 132. Inner surface 134a is formed with a radius that matches the inside of the hole 18a in the pull tab 18. This distributes the pulling force uniformly around the hole and prevents damaging of the pull tab 18 during pulling. The outer surface 134b of the bend portion 134 is curved and forms an angle 138 with the top surface 132b of the flat portion 132. In one example angles 137 and 138 are 40 and 60 degrees, respectively. Extension 136 extends from the bend portion 134, points towards the handle 110 and is parallel to the flat portion 132. In one example it has a length of 2.0 mm and a thickness of 0.8 mm. Extension 136 helps prevent slippage of the hook out of the hole 18a. The inner surface of 136 forms an angle with the inner surface 134a, which facilitates pickup of the hole 18a by the hook 130.

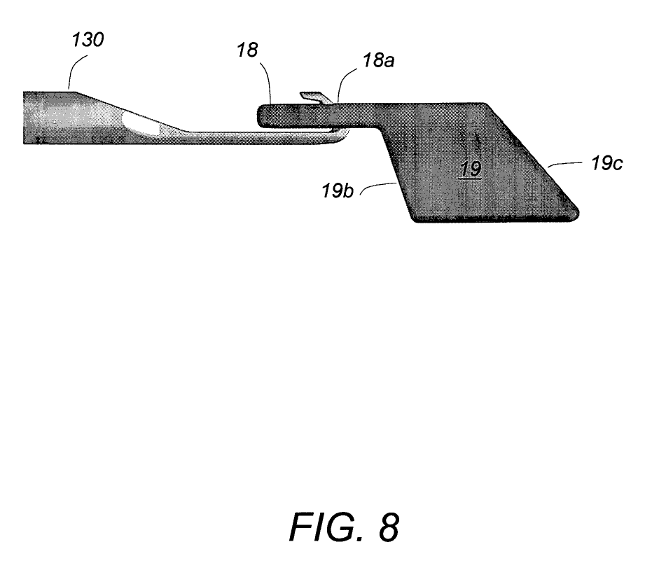

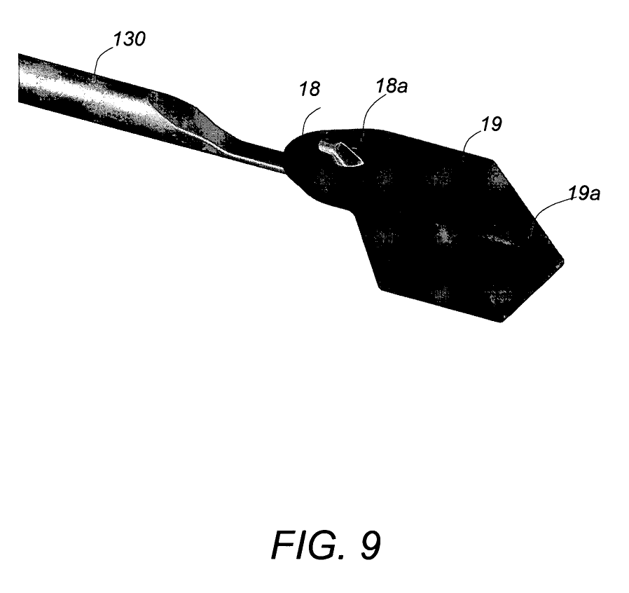

Referring to FIG. 8 and FIG. 9, the flat portion 132 of the hook end 130 is placed either underneath or on top of the pull tab 18 of the gastric band buckle and the bend portion is inserted into the hole 18a thereby engaging the pull tab. The bend portion 134 together with the extension 136 and the flat portion 132 form a C-shaped hook that grasps securely the pull tab 18. The risk of unintentional disengagement is very low. While holding the buckle end 19 securely with the hook end 130 a pusher instrument (not shown) is used to push the tube 14 of the gastric band 10 through the buckle conduit 19a. The triangular shaped member 13a of the tail portion 13 interlocks with the buckle conduit 19a and prevents the tube 14 from slipping backwards. The hook instrument and the pusher instrument apply opposing forces on the gastric band 10 in order to cinch and lock the band closed.

Several embodiments of the present invention have been described. Nevertheless, it will be understood that various modifications may be made without departing from the spirit and scope of the invention. Accordingly, other embodiments are within the scope of the following claims.

What is claimed is:

1. An endoscopic surgical hook tool used in minimally invasive surgery for grasping and tightening a ligature band, wherein said ligature band comprises an elongated strap having a buckle end and a distal end, said buckle end having an aperture and a pull tab having a hole thereon and wherein said elongated strap is configured to encircle an internal organ and said distal end is configured to pass through and lockingly engage said aperture thereby tightening said ligature band around said internal organ, the hook tool comprising:
an elongated shaft having a hook end and a handle, wherein said hook end is configured to engage said hole and pull said pull tab in a first direction while said distal end is threaded through said aperture and pushed opposite to said first direction; and
wherein said hook end comprises a flat portion and a bend portion extending from said flat portion and wherein said flat portion is configured to slide along a flat surface of said pull tab and said bend portion has an inner surface radius matching a radius of said hole.

2. The endoscopic surgical hook tool of claim 1 wherein said elongated shaft is inserted through a cannula into a patient's body and is dimensioned to enter one end, pass through and extend beyond the other end of said cannula.

3. The endoscopic surgical hook tool of claim 1 wherein said handle is configured to provide tactile control of said hook end orientation.

4. The endoscopic surgical hook tool of claim 3 wherein said handle comprises a cylindrical body having first and second side indentations opposite to each other and a thumb indentation on a top surface of said cylindrical body, said thumb indentation being aligned with said bend portion of said hook end.

5. The endoscopic surgical hook tool of claim 1 wherein said hook end further comprises an extension portion extending from said bend portion, running parallel to said flat portion and pointing towards said handle.

6. The endoscopic surgical tool of claim 1 wherein said internal organ is selected from a group consisting of stomach, artery, intestines, heart, lung, pancreas, kidney, bone and liver.

7. An endoscopic surgical hook tool used in minimally invasive surgery for grasping and pulling an elongated strap having a pull tab, the hook tool comprising:
an elongated shaft having a hook end and a handle; and
wherein said hook end comprises a flat portion, a bend portion extending from said flat portion and an extension portion extending from said bend portion and wherein said flat portion is configured to slide along a flat surface of said pull tab and said bend portion is configured to engage and grasp a hole on said pull tab and wherein said bend portion has an inner surface radius matching a radius of said hole.

8. The endoscopic surgical hook tool of claim 7 wherein said handle is configured to provide tactile control of said hook end orientation.

9. The endoscopic surgical hook tool of claim 7 wherein said elongated shaft is inserted through a cannula into a patient's body and is dimensioned to enter one end, pass through and extend beyond the other end of said cannula.

10. The endoscopic surgical hook tool of claim 7 wherein said bend portion, runs parallel to said flat portion and points towards said handle.

11. An endoscopic surgical instrument used in minimally invasive surgery for grasping and tightening a ligature band around an internal organ, wherein said ligature band comprises an elongated strap having a buckle end and a distal end, said buckle end having an aperture and a pull tab having a hole thereon and wherein said elongated strap is configured to encircle said internal organ and said distal end is configured to pass through and lockingly engage said aperture thereby tightening said ligature band around said internal organ, the instrument comprising:
a hook tool configured to engage said hole and pull said pull tab in a first direction while said distal end is threaded through said aperture and pushed opposite to said first direction;
a pusher tool configured to engage a protuberance of said distal end and push said distal end;
wherein said hook tool comprises a hook having a flat portion and a bend portion extending from said flat portion and wherein said flat portion is configured to slide along a flat surface of said pull tab and said bend portion has an inner surface radius matching a radius of said hole.

12. The endoscopic surgical instrument of claim 11 wherein said hook tool and said pusher tool apply opposing forces on said ligature band for tightening said ligature band around said internal organ.

13. A method for tightening a ligature band around an internal organ via minimally invasive surgery comprising:
providing a ligature band comprising an elongated strap having a buckle end and a distal end, wherein said buckle end has an aperture and a pull tab having a hole thereon;
inserting said ligature band into a patient's body and encircling said internal organ with said elongated strap;
inserting a surgical hook tool into the patient's body, wherein said hook tool comprises an elongated shaft having a hook end and a handle and wherein said hook end comprises a flat portion and a bend portion extending from said flat portion and wherein said flat portion is configured to slide along a flat surface of said pull tab and said bend portion has an inner surface radius matching a radius of said hole;
engaging said hole with said hook end; and
pulling said pull tab in a first direction with said hook tool while threading said distal end through said aperture and pushing said distal end opposite to said first direction.

14. The method of claim 13 wherein said elongated shaft is inserted through a cannula into a patient's body and is dimensioned to enter one end, pass through and extend beyond the other end of said cannula.

15. The method of claim 13 wherein said handle is configured to provide tactile control of said hook end orientation.

16. The method of claim 15 wherein said handle comprises a cylindrical body having first and second side indentations opposite to each other and a thumb indentation on a top surface of said cylindrical body, said thumb indentation being aligned with said bend portion of said hook end.

17. The method of claim 13 wherein said hook end further comprises an extension portion extending from said bend portion, running parallel to said flat portion and pointing towards said handle.

18. The method of claim 13 wherein said internal organ is selected from a group consisting of stomach, artery, intestines, heart, lung, pancreas, kidney, bone and liver.
